(19) United States
(12) Patent Application Publication
(10) Pub. No.: US 2026/0035751 A1
(43) Pub. Date: Feb. 5, 2026

(54) USE OF PRE-OPERATIVE BODILY FLUID AS A TUMOR GENOME REFERENCE

(71) Applicant: DROPLET BIOSCIENCES, INC., Cambridge, MA (US)

(72) Inventor: Wendy Winckler Adamovich, Melrose, MA (US)

(21) Appl. No.: 19/273,993

(22) Filed: Jul. 18, 2025

Related U.S. Application Data
(60) Provisional application No. 63/679,393, filed on Aug. 5, 2024.

(51) Int. Cl. C12Q 1/6886

(52) U.S. Cl. CPC C12Q 1/6886; C12Q 2600/112; C12Q 2600/156; G01N 2800/52

(57) ABSTRACT

The present invention provides methods for using pre-operative bodily fluid as a source for a tumor reference genome. In some embodiments, the pre-operative bodily fluid is sequenced to identify a genomic variant specific to a tumor. In some embodiments, assays are performed to detect the presence of the genomic variant specific to a tumor identified in the pre-operative bodily fluid in surgical drain fluid. In some embodiments, the bodily fluids may be blood, urine, serum, plasma, saliva, sweat, milk, mucous, semen, vaginal or urethral secretions, cerebrospinal fluid, or surgical lavage fluid.

### TECHNICAL FIELD

This invention provides methods for methods for using pre-operative bodily fluid as a tumor-informed reference library source and diagnosing and assessing disease.

### BACKGROUND

Cancer is a leading cause of death globally. Early detection, while beneficial for most cancers, is often difficult. In part, this is because many cancers first develop without presenting any specific clinical symptoms, and diagnosis only occurs when the disease has reached a stage when it is difficult to treat.

Many cancer diagnostics have focused on liquid biopsy in blood or plasma for the detection of cell-free tumor DNA. Conventional cfDNA extraction kits are available, such as the MagMax cfDNA kit (Thermo Fisher). In general, blood is of high clinical interest because of its accessibility. Unfortunately, many of these methods lack sensitivity. One reason for that is that the analytes to be detected (e.g., cfDNA) are present in very small amounts in blood. As a result, early cancer detection is often difficult. Moreover, due to the lack of sensitivity, progression of the disease and its response to therapeutic intervention are difficult to monitor.

Tissue, such as tumor tissue, generally is the most informative sample for diagnosis and prognosis of cancer. Unfortunately, tissue samples are often difficult to access and subject to limited availability, especially without performing an invasive procedure. In the context of cancer, biopsy material often gives little indication of metastatic potential.

Consequently, physicians and patients are often unable to make timely, informed decisions regarding therapeutic intervention.

### SUMMARY

The present disclosure provides methods of assessing cancer biomarkers (e.g., genomic variants, cell free DNA (cfDNA) and RNA (cfRNA), peptides, circulating tumor cells (CTC), metabolites, proteins, protein modifications, and extracellular vesicles (EV)) in pre-operative bodily fluids for monitoring therapeutic efficacy, minimal residual disease detection and monitoring metastasis and disease progression. Pre-operative bodily fluids include blood, urine, saliva, cerebrospinal fluid, stool, lymphatic drain fluid, and surgical lavage fluids.

Methods of the present disclosure are useful for diagnosing disease, predicting disease severity and progression, therapeutic selection, and detecting minimal residual disease and/or metastasis. In preferred embodiments, methods comprise sequencing nucleic acid from a body fluid sample to obtain a tumor-informed reference library that includes at least one genomic variant specific to a tumor; and performing an assay in another sample to detect the presence of the genomic variant identified in the library. In a preferred embodiment, surgical lavage fluid is the source from which the tumor-informed library is obtained and/or the sample is assayed for presence of the tumor-informed variants. The surgical drain fluid can be evaluated by sequencing methods and may be compared to the nucleic acid from the body sample from the tumor-informed reference library. In addition, a tumor-informed reference library created from nucleic acid obtained in the invention is useful to model disease progression. Libraries (e.g., tumor-informed reference libraries) of, for example, cfDNA made using methods of the present disclosure are useful for the evaluation of samples as well as for creation of a database for analysis of subsequent samples. Methods of the invention avoid the creation of a reference sample from tumor tissue.

In some preferred embodiments, the nucleic acid from a bodily fluid sample is cfDNA or ctDNA. The ctDNA obtained as described herein can be obtained from blood, urine, serum, plasma, saliva, sweat, milk, mucous, semen, vaginal or urethral secretions, cerebrospinal fluid, and surgical lavage fluid. Thus, methods of the invention are useful to create tumor-informed reference libraries that are used to track cancer progress, to detect minimal residual disease (MRD), and to predict recurrence and metastasis. Methods of the disclosure also may be used to yield quantitative information concerning cfDNA by, for example, amplifying extracted cfDNA using quantitative methods, such as qPCR.

Accordingly, the present disclosure provides methods for predicting the qualitative aspects of disease comprising rinsing a surgical site and collecting a surgical lavage fluid comprising nucleic acid prior to a tumor resection procedure; sequencing the nucleic acids to identify a genomic variant specific to the tumor. In certain embodiments, methods further comprise using the sequenced nucleic acids to assess disease state or stage. In certain embodiments, methods further comprise detecting presence of an identified genomic variant specific to the tumor in lymphatic drain fluid or surgical drain fluid post-surgery. In certain embodiments, the presence of the identified genomic variant specific to the tumor is indicative of minimal residual disease. In certain embodiments, the methods may comprise processing genomics data, including inputs as large as whole genome sequencing (WGS) to targeted panels such as an assay for tumors (such as a 596 gene panel). These types of inputs may be analyzed by a variety of methods including deep learning (e.g., CNNs, RNNs, transformers) and machine learning (e.g., SVM, gradient boosted models, logistic regression) algorithms. These outputs may be 1-dimensional. These non-linear interaction outputs from each feature set type may be merged into a single encompassing neural network architecture.

In certain embodiments, methods of the invention further include (i) diagnosing and/or staging cancer and/or (ii) selecting or evaluating a therapeutic. The disease evaluated can be any disease, including cancer, infectious disease, metabolic disease or an autoimmune disease. However, the invention has particular application in the diagnosis, prognosis, and monitoring of cancer.

Contrary to conventional thinking, lavage fluid and drain fluid, usually considered biological “waste”, are a rich source of diagnostic information. Moreover, lavage fluids and drain fluids comprising fluid originating in lymphatics and as interstitial fluid are more representative of the biomarker heterogeneity of a tumor than would be obtained from a biopsy or blood sample. The information obtained from lavage fluid and drain fluid are, therefore, informative for a diagnostic assay, whether performed on the obtained lavage fluid, obtained drain fluid, or at a subsequent time in blood or downstream lymphatic fluid.

In a preferred embodiment, the invention provides both the ability to create a tumor-informed reference library and/or a more informative personalized array of biomarkers as well as the ability to perform longitudinal monitoring of individual patients to assess recurrence, minimal residual disease and response to treatment generally.

The present invention also provides methods of library preparation from, for example, surgical lavage fluid. Libraries prepared from nucleic acid in surgical lavage fluid are informative for disease status, such as, for example, cancer status.

The invention is focused on diagnostic content in the lavage fluid or effluent originating in vivo. Lavage fluid can be obtained from routine saline rinses that are performed after a surgical site has been opened, prior to, or during, a medical intervention, such as a surgery or biopsy (e.g., tumor resection). In certain embodiments, methods of the present disclosure further comprise collecting the drain fluid within about one hour to about 48 hours post-surgery. Lavage fluid can also be obtained during treatment of a wound or interventional procedure. In certain embodiments, the methods of the present disclosures further comprise collecting the drain fluid within about one hour to about 48 hours post-surgery. A wash fluid or rinse may also be part of the lavage fluid as obtained during a medical intervention. That rinse fluid is initially captured and processed, and nucleic acid is subsequently extracted. The extracted nucleic acid may be used as tumor DNA source to create a tumor-informed reference library. Once prepared, the sequencing library or a portion thereof can be sequenced to obtain a plurality of sequence reads. The sequence reads may be in a computer-readable, digital format for processing and interpretation by computer software. Such libraries in some instances provide enhanced accuracy for diagnosing diseases or conditions and are substantially free of biological contamination.

Drain fluid can be obtained as effluent from a medical intervention, such as a surgery or biopsy. Drain fluid and lavage fluid can also be obtained during treatment of a wound or interventional procedure. The cfDNA concentration in the surgical drain fluid is assessed, for example, by electrophoresis (e.g., TapeStation System, Agilent, and/or Bioanalzyer, Agilent), and used to determine the volume of surgical drain fluid necessary for performing an assay. Then, the cfDNA from the surgical drain fluid is subject to a buffer exchange procedure followed by end repair, A-tailing, ligation of barcodes e.g., non-random unique molecular identifiers (UMIs), ligation of adaptors, and followed by PCR amplification. The buffer exchange procedure may comprise a size selection procedure. Once prepared, the cfDNA can be sequenced to obtain a plurality of sequence reads. The sequence reads may be in a computer-readable, digital format for processing and interpretation by computer software. Such methods provide enhanced accuracy for diagnosing diseases or conditions and are substantially free of biological contamination.

Tumor-informed plasma-based cfDNA assays for detection of minimal residual disease (MRD) after cancer treatment are limited by 1) sampling bias from a small sampling of the tumor, and 2) tumor heterogeneity and subclonality that develops during the course of carcinogenesis and metastasis. According to the invention, surgical drain fluid in patients undergoing cancer surgery is a good source of locoregional tumor cfDNA. The characterization and/or quantification of tumor cfDNA in the surgical drain fluid is useful to measure locoregional minimal residual disease as well as for determining the risk of recurrence. Additionally, cfDNA in drain fluid broadly captures tumor heterogeneity in a manner that is difficult or impossible with the small biopsy sample typically obtained from primary tumor tissue. Thus, drain fluid allows detection of a broader spectrum of variants, including high-risk variants, which represent the entirety of the tumor and lymph nodes metastases. Methods described herein compare cfDNA in drain fluid with nucleic acids in surgical lavage fluid to detect presence of genomic variants.

In yet another aspect, the invention provides methods for determining MRD. Tumor DNA extracted from bodily fluids (e.g., lavage fluid) using methods of the disclosure is sequenced and informative or potentially-informative genomic variants are identified. A tumor-informed reference library is obtained from the sequenced tumor DNA with identified genomic variants. Assays on surgical drain fluid are performed to detect presence of the genomic variants identified in the tumor-informed reference library. The presence and detection of genomic variants identified in the tumor-informed reference library in the surgical drain fluid is then used to monitor the patient for recurrence, disease progression, response to treatment and other clinical signs over a period of time at the discretion of the clinician. In certain embodiments, the bodily fluid sample comprises at least a wash fluid used in surgical lavage at a surgical incision made to remove a tumor, obtained prior to removal of the tumor. In certain embodiments, the methods of the present disclosure further comprise providing a report indicating the presence of minimal residual disease when the genomic variant is detected in the drain fluid. Suitable assays include, for example, nucleic acid sequencing, PCR, quantitative PCR, digital droplet PCR, Western blot target capture, proteomics, metabolomics, nucleic acid expression analysis, and antibody screening. For example, assays may include whole genome sequencing, next generation DNA sequencing, next generation RNA sequencing, multiplex PCR, methylation analysis, droplet PCR, droplet cell separation, or any combination thereof.

An advantage of this aspect of the invention is that a tumor-informed reference library is constructed, at least in part, from nucleic acids obtained in lavage fluid and allows for personalized surveillance monitoring of a patient. As discussed above, a tumor-informed reference library based on lavage fluid incorporates variants that may be missing in tumor biopsy which, by definition, may not be representative of overall tumor heterogencity. As an example, a biopsy may capture about 1 mm of, for example, a 5 cm tumor. Thus, any variants not captured in that sample will not be included in any subsequent analysis. Lavage fluid provides a more representative sample of the heterogeneity of a tumor, as lavage fluid is not confined to only a portion of the tumor. Thus, a tumor-informed reference library based on lavage fluid provides a representative and more informative assessment of tumor heterogeneity, which allows for more precision in patient longitudinal monitoring.

In general, lavage and drain fluid can be obtained as effluent from a medical intervention, such as a surgery or biopsy. Drain fluid and lavage fluid can also be obtained during treatment of a wound or interventional procedure. Methods taught herein provide sensitive and specific diagnostics that allow assessment of disease status, staging, and progression; as well as aiding in therapeutic selection and assessment of therapeutic efficacy. In certain embodiments, the surgery is a cancer surgery. In some embodiments the cancer surgery is selected from a resection surgery, a dissection surgery, an excision surgery, and any combination thereof.

As used herein, drain fluid does not imply only fluid obtained from a drain device, although that can be the case. Rather, drain fluid is intended to be fluid originating in the lymphatics and, in some cases, progressing via lymphatic channels to lymph glands and ultimately to blood. Drain fluid may comprise interstitial fluid and may be obtained as a mixture with other bodily fluids, including blood. However, the invention is focused on diagnostic content in the drain fluid or effluent originating in vivo.

The present invention is useful for evaluation of any disease biomarker. In a preferred embodiment, lavage fluid and drain fluid are sources of cell-free nucleic acid. In certain embodiments, the cell-free nucleic acid is DNA or RNA. In another embodiment, lavage and/or drain fluid are a source of circulating tumor DNA (ctDNA), tumor cells, ratios of ctDNA to cells, mutations associated with cancer, oncogenes and the like. The invention is also useful for the assessment of diseases other than cancer, including infectious diseases, autoimmune diseases, endocrine diseases and the like.

Accordingly, the present invention provides methods for disease diagnosis comprising the steps of obtaining a lavage fluid sample, extracting nucleic acid from the lavage fluid sample, conducting a size selection procedure to isolate cell-free nucleic acid in the lavage fluid sample, sequencing the cell-free nucleic acid from the lavage fluid sample to obtain a tumor-informed reference library that includes at least one genomic variant specific to a tumor, obtaining a drain fluid sample, extracting nucleic acid from the drain fluid sample, conducting a size selection procedure to isolate cell-free nucleic acid in the sample, and detecting the presence of the at least one genomic variant specific to a tumor in the cell-free nucleic acid in the drain fluid sample. In certain embodiments, methods of the invention further include (i) diagnosing and/or staging cancer and/or (ii) selecting or evaluating a therapeutic. In certain aspects, the cell-free nucleic acid is isolated without a prior step of extracting nucleic acid from the sample. The disease evaluated can be any disease, including cancer, infectious disease, metabolic disease or an autoimmune disease. However, the invention has particular application in the diagnosis, prognosis, and monitoring of cancer.

In a certain embodiment, the method includes repeating the extracting step at least once. In another embodiment, the cfDNA percentage and/or quantity may be assessed after each extraction or size selection round, and the extraction and/or size selection steps are repeated until the desired percentage and/or quantity of cfDNA is achieved. The desired percentage and/or quantity of cfDNA may correspond to a target detection threshold. In certain embodiments, the desired percentage of cfDNA is at least about 70%. In certain embodiments, the desired quantity of cfDNA is at least about 600 ng. The size selection rounds may comprise the addition of beads. In addition, the invention may further comprise treatment with Proteinase K prior to the addition of beads.

According to the present disclosure, conventional plasma cfDNA extraction kits are not suitable as the sole extraction method for cfDNA in surgical drain fluid. Surgical drain fluid differs in many aspects from plasma, including the presence of a high concentrations of cfDNA as well as genomic DNA. The present disclosure provides methods in which cfDNA isolation is enhanced through the combination of extraction and size exclusion. The present invention further provides methods direct isolation of cfDNA from drain fluid without the need for a prior nucleic acid extraction step. Such methods include steps to minimize gDNA include bead purification to size select the DNA fragments in a cfDNA sample. In some embodiments, bead purification comprises the use of magnetic beads. In some embodiments, the ratio of beads to the drain fluid can be about 0.1×, 0.2×, 0.3×, 0.4×, 0.5×, 0.6×, 0.7×, 0.8×, 0.9×, or 1×.

Additional aspect and advantages of the invention are provided below in the detailed description thereof.

### DETAILED DESCRIPTION

The present disclosure provides methods for using pre-operative bodily fluids as a source of tumor nucleic acid to construct a tumor-informed reference library. The methods of the present disclosure further comprise using drain fluid obtained from medical procedures (e.g., medical interventions such as surgeries, biopsies, catheterizations, dissections, intubations and the like) to assess diagnostic biomarkers indicative of disease.

Biomarkers are selected from nucleic acid, protein, bacteria, cells, and viruses. In preferred embodiments, the cell is a cancer cell. However, biomarkers for use in the invention may be any known biomarker for disease. For example, the biomarker may be a nucleic acid (DNA, any RNA species), a protein, or any other molecule or compound. The origin of the biomarker may be an organism, for example a bacterium, a fungus, an animal cell, a tumor cell, a protozoan cell, or a virus. The biomarkers may include mutations (e.g., genomic variants) specific to a tumor, such that higher numbers of tumor mutations predict a poor probability of survival.

According to certain embodiments of the present disclosure, conventional plasma cfDNA extraction methods are not suitable and are ineffective as the sole extraction method for cfDNA from drain fluid. Instead, cfDNA from drain fluid is subjected to a separate size exclusion step after extraction.

Drain fluid for use in the methods of the present disclosure may be obtained by any known method. For example, the drain fluid may be obtained passively as the effluent from a medical procedure. Alternatively, one may use a catheter or a drain port for actively or passively collecting fluid. Suction drainage, for example using a vacuum, may also be used to obtain fluids during a surgical procedure. The surgical drain fluid may be collected by using a syringe, pipet, or catheter, for example a Jackson-Pratt (JP) drain. The drain fluid may be collected in or transferred to a container, for example a sample vessel, such as a vial, flask, or ampule, suitable for the sterile collection of medical specimens. Surgical fluid may also be collected from biohazard waste containers, for example a suction canister, filled during a procedure or diverted from a biohazard waste container during a surgical procedure. Sample may be obtained by irrigating a surgical wound. Irrigating fluid may comprise water, saline, antibiotic solutions, antiseptic agents, or a combination thereof.

Lavage fluid for use in methods of the present disclosure may be obtained by any known method. For example, lavage fluid may be obtained passively as the effluent from a medical procedure. Alternatively, one may use a catheter or a drain port for actively or passively collecting fluid. Suction drainage, for example using a vacuum, may also be used to obtain fluids during a surgical procedure. The surgical lavage fluid may be collected by using a syringe, pipet, or catheter, for example a Jackson-Pratt (JP) drain. The lavage fluid may be collected in or transferred to a container, for example a sample vessel, such as a vial, flask, or ampule, suitable for the sterile collection of medical specimens. Lavage fluid may also be collected from biohazard waste containers, for example a suction canister, filled during a procedure or diverted from a biohazard waste container during a surgical procedure. Sample may be obtained by irrigating a surgical wound. Irrigating fluid may comprise water, saline, antibiotic solutions, antiseptic agents, or a combination thereof.

Surgical drain fluid may be collected from any surgical procedure. For example, the surgery may comprise an open surgical procedure or an endoscopic procedure. The surgical procedure may comprise an invasive procedure. The surgical procedure may comprise a resection, biopsy, dissection, or excision. The surgical procedure may be a thoracentesis. The surgical procedure may be a minimally invasive procedure, such as, for example, a stent placement. The surgical procedure may be a procedure that is not a related to a disease being diagnosed. Thus, the invention applies to any disease condition and the drain fluid may be from an unrelated interventional procedure. Drain fluid may be obtained at any time during or following an interventional procedure. For example, drain fluid may be collected at the time of intervention and then periodically over the course of hours, days or weeks. Surgical drain fluid is preferably collected as the drainage from a surgical site. In preferred embodiments, the drain fluid is obtained from a site proximal to the tumor. For example, during or after a lymphadenectomy, the site of lymph node dissection is often at the closest lymph node to a tumor (e.g., that has been detected by, e.g., x-ray or other means). The surgical drain fluid may include that fluid that collects in the body of the subject at a site of surgery. The surgical drain fluid may be collected into a collection bulb or vessel. While the compositions of drain fluid may change over time during and after the surgery, typically it will always contain lymph (very early, e.g., during the incision, it may be predominantly blood). Starting at the time of surgery, it may be found that the amount of lymph present increases as the surgical site heals. The surgical drain fluid may be obtained via a drain (such as a JP drain) that includes a tube positioned to collect the drain fluid from a surgical site where a lymph node has been removed.

Surgical lavage fluid may be collected from any surgical procedure. For example, the surgery may comprise an open surgical procedure or an endoscopic procedure. The surgical procedure may comprise an invasive procedure. The surgical procedure may comprise a resection, biopsy, dissection, or excision. The surgical procedure may be a thoracentesis. The surgical procedure may be a minimally invasive procedure, such as, for example, a stent placement. The surgical procedure may be a procedure that is not a related to a disease being diagnosed. Thus, the invention applies to any disease condition and the lavage fluid may be from an unrelated interventional procedure. Lavage fluid may be obtained at any time prior to or during an interventional procedure. For example, Lavage fluid may be collected prior to or at the time of intervention.

As discussed above, surgical drain fluids have informative genomic content. Methods to minimize gDNA contamination include bead purification to size select the DNA fragments in a cfDNA sample. In some embodiments, isolating cfDNA is done in such a manner as to maximize the recovery of short fragments (<100 base pairs), as the composition of short fragments differs more between healthy and disease states than the composition of longer fragments. In some embodiments, the cfDNA fragments are subjected to a size selection to retain only cfDNA fragments having a length between an upper bound and a lower bound. In some embodiments, the upper bound is about 200, about 190, about 180, about 170, about 160, about 150, about 140, about 130, about 120, about 110, about 100, about 90, about 80, about 70, about 60, or about 50 base pairs and the lower bound is about 20, about 25, about 30, about 35, about 36, about 40, about 45, about 50, about 60, about 70, about 80, about 90, about 100, about 110, or about 120 base pairs. In some embodiments, the lower bound is 36 and the upper bound is 100. In some embodiments, the beads are magnetic beads. In some embodiments, the ratio of beads to the drain fluid can be 0.1×, 0.2×, 0.3×, 0.4×, 0.5×, 0.6×, 0.7×, 0.8×, 0.9×, and 1×. In some embodiments, the ratio of magnetic beads may be added in staggered steps, e.g., where 0.4× beads are added, and then separated and an additional 0.1× beads is added to the supernatant.

In various aspects, nucleic acids may be identified and quantified using methods known in the art. Suitable assays include, for example, nucleic acid sequencing, PCR, quantitative PCR, digital and droplet PCR. Sequencing may be performed by various methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Nucleic acid molecules may advantageously be amplified prior to sequencing. Amplification may comprise methods for creating copies of nucleic acids by using thermal cycling to expose reactants to repeated cycles of heating and cooling, and to permit different temperature-dependent reactions (e.g., by Polymerase chain reaction (PCR). Any suitable PCR method known in the art may be used in connection with the presently described methods. Non limiting examples of PCR reactions include real-time PCR, nested PCR, multiplex PCR, quantitative PCR, or touchdown PCR.

Tumor-informed plasma-based cfDNA assays for detection of minimal residual disease (MRD) after cancer treatment are limited by 1) sampling bias from a small sampling of the tumor, and 2) tumor heterogeneity and subclonality that develops during the course of carcinogenesis and metastasis. Further, traditional tumor-informed approaches for detection of minimal residual disease (MRD) after cancer treatment (e.g., neoadjuvant therapies) are limited by 1) the low availability of tumor tissue, 2) low quality DNA from tumor tissue, and 3) insufficient amount of tumor cells contained within the tumor tissue. According to the invention, surgical drain fluid in patients undergoing cancer surgery is a good source of locoregional tumor cfDNA. The characterization and/or quantification of tumor cfDNA in the surgical drain fluid is useful to measure minimal residual disease as well as for determining the risk of recurrence. Additionally, cfDNA in drain fluid broadly captures tumor heterogeneity in a manner that is difficult or impossible with the small biopsy sample typically obtained from primary tumor tissue. Thus, drain fluid allows detection of a broader spectrum of variants, including high-risk variants, which represent the entirety of the tumor and lymph nodes metastases. Methods described herein compare nucleic acids in drain fluid with nucleic acids in surgical lavage fluid to detect presence of genomic variants.

In yet another aspect, the invention provides methods for determining MRD. Tumor DNA extracted from bodily fluids (e.g., lavage fluid) using methods of the disclosure is sequenced and informative or potentially-informative genomic variants are identified. A tumor-informed reference library is obtained from the sequenced tumor DNA with identified genomic variants. Assays on surgical drain fluid are performed to detect presence of the genomic variants identified in the tumor-informed reference library. The presence and detection of genomic variants identified in the tumor-informed reference library in the surgical drain fluid is then used to monitor the patient for recurrence, disease progression, response to treatment and other clinical signs over a period of time at the discretion of the clinician. In certain embodiments, the bodily fluid sample comprises at least lymph and a wash fluid used in surgical lavage at a surgical incision made to remove a tumor, obtained prior to removal of the tumor. In certain embodiments, the methods of the present disclosure further comprise providing a report indicating the presence of minimal residual disease when the genomic variant is detected in the drain fluid. Suitable assays include, for example, nucleic acid sequencing, PCR, quantitative PCR, digital droplet PCR, Western blot target capture, proteomics, metabolomics, nucleic acid expression analysis, and antibody screening. For example, assays may include whole genome sequencing, next generation DNA sequencing, next generation RNA sequencing, multiplex PCR, methylation analysis, droplet PCR, droplet cell separation, or any combination thereof. ctDNA may indicate the presence of MRD following surgical resection and predict risk of recurrence with a high degree of precision. In embodiments, a pre-operative bodily fluid sample is obtained at or prior to the time of the surgical procedure at a first time point, and a biomarker indicative of disease is identified (e.g., genomic variant). Further, the method contemplates obtaining a surgical drain fluid sample at a second time point and identifying in the surgical drain fluid sample the presence or absence of the biomarker indicative of disease (e.g., genomic variant). In embodiments, the second sample is obtained from the patient after the patient has undergone treatment. Therefore, the presence or absence of the biomarker is an indication of treatment efficacy.

Thus, identifying a difference in the presence or absence of a biomarker related to the cancer removed from the patient by comparing biomarker(s) in lavage fluid samples and drain fluid samples, may be indicative of minimal residual disease.

MRD can be defined as cancer persisting in a patient, after treatment, which may not be detected with current medical imaging modalities and is, therefore, an occult stage of cancer progression. Methods of the invention use biomarkers from effluent to evaluate MRD to see if the cancer treatment is working and to guide further treatment plans. Methods of the invention use liquid biopsy approaches based on the detection of small numbers of circulating tumor cells (CTCs) or minute amounts of circulating cell-free tumor DNA (ctDNA) to detect MRD in patients with various malignancies. Using methods of the invention, monitoring CTCs and ctDNA during post-surgical follow-up assessments enables detection of disease relapse, or determines the success of surgical outcomes, earlier than is possible with current radiological imaging procedures. Further characterization of CTCs or ctDNA provides insights into the molecular evolution of MRD during tumor progression with implications for therapeutic interventions to delay or prevent metastatic relapse.

Methods of the invention may use cfDNA or ctDNA as biomarkers for surgical success, disease recurrence, and/or determining the efficacy of treatment. As an example, the presence of ctDNA is associated with a worse overall survival and disease recurrence. Methods of the invention may use the drain fluid sample to monitor for ctDNA and indications of cancer recurrence. As another example, cell-free DNA (cfDNA) is significantly higher in patients with cancer than in healthy controls or patients with benign lesions. Detected ctDNA and increased cfDNA are associated with decreased survival. Thus, methods of the invention can detect cfDNA that may be associated with cancer.

Sequencing may be performed on any suitable platform including, for example, Sanger sequencing, so-called Next Generation Sequencing (NGS) technologies such as those instruments and methods offered by ILLUMINA, ROCHE, or ULTIMA, single molecule sequencing using instruments or technologies offered by PACBIO or OXFORD NANOPORE, others, or combinations thereof. The sequencing may be whole genome or target specific genetic segments, genes, mutations, or panels of mutations.

Common sequencing instruments yield sequence reads, e.g., in the FASTA or FASTQ format. The reads from nucleic acids obtained from surgical lavage fluid may be aligned to a reference, such as the “hg37” human genome reference, and the alignments can be reported as, and saved as, a sequence alignment map (SAM) or binary alignment map (BAM) file. From the comparison to the reference genome, places where the sequence reads do not match (vary from) the reference may be identified as “genomic variants” (aka mutations), which is sometimes reported and stored in a variant call format (*.vcf) file, aka a VCF file. Such method steps may proceed with steps and formats as described in U.S. Pat. No. 8,209,130, incorporated by reference. Mutations (or variants) may be read or counted from the VCF files. The identified genomic variants may be further aligned to nucleic acids obtained from surgical drain fluid. Detection of the presence of an identified genomic variant specific to a tumor in surgical drain fluid is indicative of minimal residual disease. Genomic variants include any alteration in a gene or gene product (e.g., RNA, peptide, and/or protein) and refers to, e.g., the presence of a mutation or mutations within the gene or gene product, an alteration in copy number of the gene or gene product, or a translocation of the gene or gene product. The genomic variant may affect the integrity, sequence, structure, amount, or activity of the gene or gene product as compared to the wild-type gene. Certain exemplary genomic variants include those relating to the nucleic acid sequence of all or a portion of the genome (e.g., nucleotide polymorphism, indel, sequence rearrangement, mutational frequency, and/or chromosomal translocation), the copy number of one or more particular nucleotide sequences within the genome (e.g., copy number, single chromosome or entire genome ploidy, and/or allele frequency fractions), and the expression profile of the organism's genome (e.g., gene expression levels, isotype expression levels, and/or gene expression ratios). In some embodiments, the genomic variant comprises a gene fusion. In some embodiments, genomic variant is selected from the group comprising single nucleotide variants (SNVs), copy number variants or variations (CNVs)/aberrations, insertions or deletions (indels), truncation, gene fusions, transversions, translocations, frame shifts, duplications, repeat expansions, and epigenomic variants (one or more of methylation, chromatin structure, etc.).

An advantage of this aspect of the invention is that a tumor-informed reference library is constructed, at least in part, from nucleic acids obtained in lavage fluid and allows for personalized surveillance monitoring of a patient. As discussed above, a tumor-informed reference library based on lavage fluid incorporates variants that may be missing in tumor biopsy which, by definition, may not be representative of overall tumor heterogeneity. As an example, a biopsy may capture about 1 mm of, for example, a 5 cm tumor. Thus, any variants not captured in that sample will not be included in any subsequent analysis. Lavage fluid provides a more representative sample of the heterogeneity of a tumor, as lavage fluid is not confined to only a portion of the tumor. Thus, a tumor-informed reference library based on lavage fluid provides a representative and more informative assessment of tumor heterogeneity, which allows for more precision in patient longitudinal monitoring.

In another aspect, the invention provides a method of monitoring efficacy of a treatment, the method comprising the steps of: obtaining, as a first sample, pre-operative bodily fluid sample from a patient prior to or during a surgical procedure; obtaining, as a second sample, surgical drain fluid from the patient after the surgical procedure; identifying in the sample a difference in the first sample and the second sample in a biomarker indicative of disease; and determining the efficacy of treatment based on the difference.

The first sample may be obtained during or prior to the time of the surgical procedure. The second or multiple subsequent samples may be obtained while the patient is undergoing a separate, surgical procedure occurring at a different time point than the first surgical procedure. The collection times are specific points in time wherein the second fluid sample is taken at a later point in time than the first fluid sample. In embodiments, the second sample, or subsequent samples, is taken post-treatment. A minimum of a first and second sample at first and second time points are collected. However, multiple subsequent samples at corresponding multiple subsequent timepoints may also be collected.

Methods of the invention comprise identifying a difference, or differences, between the first sample and the second sample. Treatment efficacy, as well as assessing disease progression, severity, staging, prognosis or diagnosis may be evaluated based on the difference or differences. Identified differences include presence or absence of one or more biomarkers, changes in quantity, amount, weighted amount, quality, heterogencity (both in terms of genomic heterogeneity and morphologic heterogeneity), velocity of change, and/or accumulated changes over time between two or more measurements. For example, differences may include the rate of accumulation of a biomarker, an amount of tumor cells, or an amount of cell-free DNA or RNA. The difference may also be the fragment size of cell-free DNA or RNA wherein a decrease in average fragment size between the first effluent sample at the first time point and the second effluent sample at a second time points is indicative of disease progression. The difference may also be measured as presence/absence or positive/negative for the presence of a biomarker or set of biomarkers.

If a biomarker or other quantity of interest has increased in concentration between the first sample at the first time point, and the second sample at the second time point, a difference is identified which is an indication of disease progression or advancement, or that treatment is not efficacious. For example, in cancer, disease progression is often defined by cancer that continues to grow or spread. Progression-free survival (PFS) for patients with cancer is the length of time during and after treatment of a disease that a patient lives with the disease while the disease does not worsen. For clinical trials, measuring the progression-free survival (PFS) is one way to see how well a new treatment works. Therefore, the information obtained from the difference identified between the first sample and the second sample may be used to assess disease progression and treatment efficacy. Thus, methods of the invention are useful to identify effective therapeutics and to assess the efficacy of treatments.

In one embodiment, methods of the invention assess a rate of accumulation of a biomarker as indicative of disease status or progression. The rate of accumulation of the biomarker may be represented as the gradual acquisition of a mass or quantity over time, or the progressive increase in concentration over time. According to methods of the invention the rate of accumulation or decrease of biomarkers in a sample of effluent may be indicative of disease severity and whether a disease is progressing or regressing. For example, if the effluent is measured at multiple time points, it is possible to calculate a slope of biomarker accumulation. The steepness of the slope is indicative of the velocity of change (either negative or positive). In the same way, the area under the curve resulting from multiple measurements in the drain fluid is indicative of disease progression or regression. In assessing disease progression, the rate of accumulation may offer information about the effect of therapeutic intervention. In addition, the rate of accumulation may identify different tumor types that are amenable to certain therapies and may also identify patients who will benefit from such treatment. The concentration, mass or quantity of a biomarker in the first sample and again in the second or subsequent samples may be measured. The difference in concentration, mass or quantity of a biomarker or plurality of biomarkers over time is used to calculate the rate of accumulation of the biomarker.

As noted above, in embodiments, assessing efficacy of treatment or disease progression may be evaluated by identifying the difference in amounts of tumor cells in first and second samples. The exact nature of the cell being measured may vary. For example, tumor cells may be, for example, circulating tumor cells, tumor-derived exosomes, or circulating tumor nucleic acids. The invention also contemplates detecting circulating tumor nucleic acids released from tumor cells. Tumor cells in the sample are quantified using any suitable detection technologies with or without enrichment, including but not limited to, fluorescence, surface-enhanced Raman scattering, or electrical impedance. The invention provides for assessing disease progression by identifying differences in an amount of tumor cells in first and subsequent effluent samples. Differences may be used to monitor disease progression, diagnosis, chemotherapeutic efficacy, and may also provide insight into the biology of metastatic cancer.

As discussed above, the surgical procedure may be any procedure. In non-limiting examples, the surgery may be for colorectal, small intestine, ovarian, rectal, anal, bladder or prostate cancer (or any other cancer). The waste fluid may or may not be in the proximity to the surgical site. In certain embodiments, the surgery is a cancer surgery. In some embodiments the cancer surgery is selected from a resection surgery, a dissection surgery, an excision surgery, and any combination thereof.

Methods of the invention are also useful for assessment of therapeutic efficacy. In this aspect, cfDNA in lavage fluid is used to obtain a tumor-informed reference library and establish a baseline. Then cfDNA in drain fluid of the patient is monitored through a course of treatment to determine the effect of the treatment on reducing or increasing the presence of tumor-associated cfDNA in drain fluid over time as a proxy for successful treatment of the primary tumor and/or metastasis.

Methods of the invention also contemplate assessment of pharmaceutical efficacy. According to the invention, accumulation of ctDNA in drain fluid after therapy, compared to ctDNA in pre-operative bodily fluid (e.g., lavage fluid), is indicative that the therapy is effective, as an increase in tumor DNA in drain fluid after therapy is indicative of the induction of cell death in the tumor. Thus, real-time measurement during therapy is indicative of therapeutic efficacy. According to methods of the invention the rate of accumulation or decrease of cfDNA in drain fluid, compared to ctDNA in pre-operative bodily fluid (e.g., lavage fluid), is indicative of disease severity and whether a disease is progressing or regressing. For example, if drain fluid is measured at multiple time points, it is possible to calculate a slope of cfDNA accumulation. The slope is indicative of the velocity of change (either negative or positive). In the same way, the area under the curve resulting from multiple measurements in the drain fluid is indicative of disease progression or regression.

In one aspect, a method for detecting minimal residual disease in a subject undergoing a cancer surgery is disclosed. The method includes obtaining a pre-operative bodily fluid sample (e.g., lavage fluid) from the subject prior to, or during, cancer surgery. The method further includes obtaining a drain fluid sample post-surgery. The method also includes isolating an amount of tumor-associated genetic material from the pre-operative bodily fluid sample and/or drain fluid sample, sequencing the amount of tumor-associated genetic material to detect and quantify at least one tumor-associated mutation or genomic variant in the amount of tumor-associated genetic material, and providing the at least one quantity of the at least one tumor-associated mutation or variant to a practitioner. The at least one tumor-associated mutation or genomic variant is indicative of minimal residual disease in the subject. In some aspects, the amount of tumor-associated genetic material includes cell-free DNA, RNA, proteins, exosomes, and any combination thereof. In some aspects, isolating the amount of tumor-associated genetic material from the sample further includes filtering the sample, centrifuging the sample, contacting the sample with a chromatography medium, and any combination thereof. In some aspects, the method further includes selecting an additional treatment based on the presence and/or quantity of the at least one tumor-associated mutation or genomic variant. In some aspects, the additional treatment is selected from radiotherapy, chemotherapy, follow-up surgery, active surveillance with imaging, and any combination thereof. In some aspects, the tumor-associated genetic material is produced by a plurality of cancer cells. In some aspects, the plurality of cancer cells are selected from one of oropharyngeal cancer cells, lung cancer cells, breast cancer cells, melanoma cells, colon cancer cells, thyroid cancer cells, prostate cancer cells, ovarian cancer cells, testicular cancer cells, penile cancer cells, cervical cancer cells, anal cancer cells, brain cancer cells, liver cancer cells, pancreatic cancer cells, and testicular cancer cells. In some aspects, the cancer surgery is selected from a resectioning surgery, a dissection surgery, an excision surgery, and any combination thereof. In some aspects, obtaining the sample from the subject further includes capturing a surgical drainage from a drainage tube associated with the cancer surgery. In some aspects, obtaining the sample from the subject further includes capturing a surgical drainage from the drainage tube within about 24 hours of the cancer surgery. In some aspects, the tumor-associated genetic material includes cell-free DNA (cfDNA) associated with cancer cells. In some aspects, sequencing the amount of tumor-associated genetic material to detect and quantify at least one tumor-associated mutation or genomic variant further includes subjecting the sample to a sequencing method selected from next generation DNA sequencing, next generation RNA sequencing, next generation protein sequencing, PCR, Western blot, and any combination thereof.

In another aspect, a method for selecting a post-operative treatment for a cancer patient in need is disclosed. The method includes obtaining a pre-operative bodily fluid sample (e.g., lavage fluid) from the subject prior to, or during, cancer surgery. The method further includes obtaining a drain fluid sample post-surgery. The method also includes isolating an amount of tumor-associated genetic material from the pre-operative bodily fluid sample and/or drain fluid sample, sequencing the amount of tumor-associated genetic material to detect and quantify at least one tumor-associated mutation or genomic variant in the amount of tumor-associated genetic material, and providing the at least one quantity of the at least one tumor-associated mutation or variant to a practitioner. The at least one tumor-associated mutation or genomic variant is indicative of minimal residual disease in the subject. In some aspects, the amount of tumor-associated genetic material includes cell-free DNA, RNA, proteins, exosomes, and any combination thereof. In some aspects, isolating the amount of tumor-associated genetic material from the sample further includes filtering the sample, centrifuging the sample, contacting the sample with a chromatography medium, and any combination thereof. In some aspects, the method further includes selecting an additional treatment based on the presence and/or quantity of the at least one tumor-associated mutation or genomic variant. In some aspects, the additional treatment is selected from radiotherapy, chemotherapy, follow-up surgery, active surveillance with imaging, and any combination thereof. In some aspects, the tumor-associated genetic material is produced by a plurality of cancer cells. In some aspects, the plurality of cancer cells are selected from one of oropharyngeal cancer cells, lung cancer cells, breast cancer cells, melanoma cells, colon cancer cells, thyroid cancer cells, prostate cancer cells, ovarian cancer cells, testicular cancer cells, penile cancer cells, cervical cancer cells, anal cancer cells, brain cancer cells, liver cancer cells, pancreatic cancer cells, and testicular cancer cells. In some aspects, the cancer surgery is selected from a resectioning surgery, a dissection surgery, an excision surgery, and any combination thereof. In some aspects, obtaining the sample from the subject further includes capturing a surgical drainage from a drainage tube associated with the cancer surgery. In some aspects, obtaining the sample from the subject further includes capturing a surgical drainage from the drainage tube within about 24 hours of the cancer surgery. In some aspects, the tumor-associated genetic material includes cell-free DNA (cfDNA) associated with cancer cells. In some aspects, sequencing the amount of tumor-associated genetic material to detect and quantify at least one tumor-associated mutation or genomic variant further includes subjecting the sample to a sequencing method selected from next generation DNA sequencing, next generation RNA sequencing, next generation protein sequencing, PCR, Western blot, and any combination thereof.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of detecting minimal residual disease, the method comprising:
sequencing nucleic acid from a bodily fluid sample to obtain a tumor-informed reference library that includes at least one genomic variant specific to a tumor; and
performing an assay on surgical drain fluid to detect presence of the genomic variant identified in said library.

2. The method of claim 1, wherein the bodily fluid sample is selected from the group consisting of blood, urine, serum, plasma, saliva, sweat, milk, mucous, semen, vaginal or urethral secretions, cerebrospinal fluid, and surgical lavage fluid.

3. The method of claim 1, wherein the bodily fluid sample comprises at least lymph and a wash fluid used in surgical lavage at a surgical incision made to remove a tumor, obtained prior to removal of the tumor.

4. The method of claim 1, further comprising collecting the drain fluid within about one hour to about 48 hours post-surgery.

5. The method of claim 1, further comprising providing a report indicating the presence of minimal residual disease when the genomic variant is detected in the drain fluid.

6. The method of claim 1, wherein the bodily fluid sample is obtained during surgery.

7. The method of claim 1, wherein the nucleic acid is cfDNA.

8. The method of claim 1, wherein the surgical drain fluid comprises cell-free nucleic acid and the performing step comprises sequencing the cell-free nucleic acid.

9. The method of claim 1, further comprising a step of diagnosing and/or staging cancer.

10. The method of claim 1, further comprising selecting an additional treatment based on the presence of the at least one genomic variant.

11. The method of claim 10, wherein the additional treatment is selected from radiotherapy, chemotherapy, follow-up surgery, active surveillance with imaging, and any combination thereof.

12. The method of claim 1, wherein the tumor is selected from one of oral tumor, laryngeal tumor, lung tumor, bladder tumor, kidney tumor, breast tumor, melanoma cells, colon tumor, thyroid tumor, prostate tumor, ovarian tumor, testicular tumor, penile tumor, cervical tumor, anal tumor, brain tumor, liver tumor, pancreatic tumor, and testicular tumor.

13. A method for assessing surgical success comprising:
rinsing a surgical site and collecting a surgical lavage fluid comprising nucleic acids prior to a tumor resection procedure;
sequencing the nucleic acids to identify a genomic variant specific to the tumor.

14. The method of claim 13, further comprising using the sequenced nucleic acids to assess disease state or stage.

15. The method of claim 13, further comprising detecting presence of the identified genomic variant specific to the tumor in surgical drain fluid post-surgery.

16. The method of claim 15, wherein presence of the identified genomic variant specific to the tumor is indicative of minimal residual disease.

17. The method of claim 15, wherein the surgical drain fluid is obtained within about 24 hours of the tumor resection procedure.

18. The method of claim 13, wherein the tumor is selected from one of oral tumor, laryngeal tumor, lung tumor, bladder tumor, kidney tumor, breast tumor, melanoma cells, colon tumor, thyroid tumor, prostate tumor, ovarian tumor, testicular tumor, penile tumor, cervical tumor, anal tumor, brain tumor, liver tumor, pancreatic tumor, and testicular tumor.

19. The method of claim 13, wherein the nucleic acids comprise cfDNA.

20. The method of claim 15, further comprising selecting an additional treatment based on the presence of the at least one genomic variant.

21. The method of claim 20, wherein the additional treatment is selected from radiotherapy, chemotherapy, follow-up surgery, active surveillance with imaging, and any combination thereof.
